(19) United States
(12) Patent Application Publication
(10) Pub. No.: US 2026/0168002 A1
(43) Pub. Date: Jun. 18, 2026

(54) PROCESS FOR PRODUCING GALACTOOLIGOSACCHARIDES

(71) Applicant: fairlife, LLC, Chicago, IL (US)

(72) Inventors: Ghadeer Makki, Chicago, IL (US); Kamil P. Drapala, Chicago, IL (US); Shakeel Ur Rehman, Naperville, IL (US); Timothy Peter Doelman, Glencoe, IL (US)

(21) Appl. No.: 19/127,230

(22) PCT Filed: Nov. 20, 2023
(86) PCT No.: PCT/US2023/080559

§ 371 Date: May 5, 2025

Related U.S. Application Data
(60) Provisional application No. 63/384,619, filed on Nov. 22, 2022.

(51) Int. Cl. C12P 19/14; A23C 9/12; A23C 9/142; C12N 9/38

(52) U.S. Cl. CPC C12P 19/14; A23C 9/1206; A23C 9/1422; C12N 9/2471; C12Y 302/01023; A23C 2210/206

(57) ABSTRACT

Methods for making galactooligosaccharides (GOS) include (a) contacting a lactose-containing product and a first β-galactosidase enzyme to form a first composition, (b) deactivating the first β-galactosidase enzyme in the first composition, (c) adjusting a temperature of the first composition to within a range from 5 to 55° C., (d) contacting the first composition and a second β-galactosidase enzyme to form a second composition, (e) deactivating the second β-galactosidase enzyme in the second composition, and (f) subjecting the second composition to membrane filtration to form a concentrated (GOS) composition. GOS compositions can contain 0.5 to 6 wt. % lactose and 24 to 50 wt. % GOS, based on carbohydrates, and often have a weight ratio of GOS:lactose ranging from 6:1 to 45:1.

### REFERENCE TO RELATED APPLICATION

This application is being filed on Nov. 20, 2023, as a PCT International Patent Application and claims the benefit of and priority to U.S. Provisional Patent Application No. 63/384,619, filed on Nov. 22, 2022, the disclosure of which is incorporated herein by reference in its entirety.

### BACKGROUND OF THE INVENTION

The present invention relates generally to the preparation of galactooligosaccharides from lactose-containing dairy streams.

### SUMMARY OF THE INVENTION

This summary is provided to introduce a selection of concepts in a simplified form that are further described herein. This summary is not intended to identify required or essential features of the claimed subject matter. Nor is this summary intended to be used to limit the scope of the claimed subject matter.

Processes for producing galactooligosaccharides are disclosed and described herein. A representative process can comprise (a) contacting a lactose-containing product and a first β-galactosidase enzyme to form a first composition, (b) deactivating the first D-galactosidase enzyme in the first composition, (c) adjusting a temperature of the first composition to within a range from 5 to 55° C., (d) contacting the first composition and a second β-galactosidase enzyme to form a second composition, (e) deactivating the second β-galactosidase enzyme in the second composition, and (f) subjecting the second composition to membrane filtration to form a concentrated galactooligosaccharides (GOS) composition.

Galactooligosaccharides (GOS) compositions also are disclosed and described herein. A representative composition can comprise from 0.5 to 6 wt. % lactose and from 24 to 50 wt. % GOS (DP3+), based on carbohydrates. Often, the weight ratio of GOS:lactose in the composition ranges from 6:1 to 45:1.

Both the foregoing summary and the following detailed description provide examples and are explanatory only. Accordingly, the foregoing summary and the following detailed description should not be considered to be restrictive. Further, features or variations can be provided in addition to those set forth herein. For example, certain aspects can be directed to various feature combinations and sub-combinations described in the detailed description.

### BRIEF DESCRIPTION OF THE FIGURE

The following FIGURE forms part of the present specification and is included to further demonstrate certain aspects of the present invention. The invention may be better understood by reference to this FIGURE in combination with the detailed description and examples.

FIG. 1 presents a schematic flow diagram of a process for producing galactooligosaccharides (GOS) consistent with an aspect of this invention.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2^{nd} Ed (1997), can be applied, as long as that definition does not conflict with any other disclosure or definition applied herein, or render indefinite or non-enabled any claim to which that definition can be applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and/or feature disclosed herein, all combinations that do not detrimentally affect the designs, compositions, processes, and/or methods described herein are contemplated with or without explicit description of the particular combination. Additionally, unless explicitly recited otherwise, any aspect and/or feature disclosed herein can be combined to describe inventive designs, compositions, processes, and/or methods consistent with the present invention.

In this disclosure, while compositions and methods are often described in terms of “comprising” various components or steps, the compositions and methods also can “consist essentially of” or “consist of” the various components or steps, unless stated otherwise. The terms “a,” “an,” and “the” are intended to include plural alternatives, e.g., at least one, unless otherwise specified.

In the disclosed methods, the term “contacting” encompasses the combining of components in any order, in any manner, and for any length of time, unless otherwise specified. For example, the components can be blended or mixed.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein. For example, the lactose-containing product can contain from 6 to 50 wt. % lactose in aspects of this invention. By a disclosure that the amount of lactose in the lactose-containing product is from 6 to 50 wt. %, the intent is to recite that the amount of lactose can be any amount in the range and, for example, can include any range or combination of ranges from 6 to 50 wt. %, such as from 8 to 40 wt. %, from 10 to 30 wt. %, or from 10 to 20 wt. % lactose, and so forth. Likewise, all other ranges disclosed herein should be interpreted in a manner similar to this example.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods, devices, and materials similar or equivalent to those described herein can be used in the practice or testing of the invention, the typical methods, devices, and materials are herein described.

All publications and patents mentioned herein are incorporated herein by reference in their entirety for the purpose of describing and disclosing, for example, the constructs and methodologies that are described in the publications and patents, which might be used in connection with the presently described invention.

### DETAILED DESCRIPTION OF THE INVENTION

Disclosed herein are processes for producing galactooligosaccharides (GOS) from lactose-containing dairy streams. Lactose, also known as milk sugar, is a disaccharide composed of two sugar monomers: D-glucose and D-galactose interconnected with β 1-4 linkage and has the chemical structure (β-D-galactopyranosyl-(1→4)-α-D-glucopyranose). Lactose is a component of milk in all mammals ranging in concentration between 2 and 10 wt. %. Bovine milk is the most widely consumed milk in the US with an average lactose level of 4.8 wt. %. Lactose is considered a by-product of many dairy processing operations such as cheese, yogurt, and membrane filtration. The relatively low sweetness index and low solubility of lactose limits its use as a sweetener. While being a valuable nutrient, especially for infants, intolerance to lactose represents an added challenge to its use as a sweetener. On average, approximately 65% of the population is reported as being lactose intolerant to some extent. Lactose intolerance implies that an individual's ability to digest lactose is compromised due to the deficiency of lactase enzyme. Moreover, the high oxygen demand of lactose makes lactose disposal an additional challenge. All these factors demonstrate a need for alternatives in order to maximize lactose utilization. Bioconversion of lactose into glucose-galactose syrup as a sweetener is one alternative. However, the relatively high cost and the bitter taste of the syrup limits its application.

Another alternative is to create value-added ingredients from underutilized lactose streams, such as galactooligosaccharides (GOS), lactulose, polylactose, and lactobionic acid. GOS represent an emerging class of natural dietary fibers with a wide range of variability in terms of composition. GOS compositions can have between 2 to 20 molecules of galactose and 1 molecule of glucose. Galactooligosaccharides are naturally present in breast milk, garlic, onion, soybean, and chicory roots.

GOS prebiotic activity has been a subject of many studies relating to the health benefits. Prebiotic generally is defined as a non-digestible food ingredient that beneficially affects the host by selectively stimulating the growth and/or activity of one or more of a limited number of bacteria in the colon, and thus improves host health. GOS are acid-resistant and thus can withstand the acidic environment of the stomach without digestion with around 90% of the ingested content reaching the gut. The human gut microbiota is complex in nature, being composed of more than 500 bacterial species. These species have a diverse range of genes allowing them to ferment non-digestible carbohydrates and produce a wide range of bioactive compounds including short chain fatty acids (SCFAs). SCFAs have a direct impact on the function of intestinal mucosa, serving as an energy source and modulating water and mineral absorption. Studies have shown that GOS stimulates specifically the growth of bifidobacteria and lactobacilli, which are predominant in the human gut. Other health benefits linked to GOS consumption include enhanced bowel function, reducing the incidence of gut inflammation, and modulating the immune response to allergy.

Traditionally, GOS are produced through acid hydrolysis of lactose that results in the formation of complex mixture of di-saccharides and tri-saccharides together with anhydro-sugars. The harsh processing parameters as well as complex nature of products limit the feasibility of scaling up this process. GOS are now often produced through the catalytic activity of glycoside hydrolases, also known as β-galactosidases. These enzymes operate through two steps: hydrolysis and transgalactosylation. In the hydrolysis step, lactose is cleaved into its component monosaccharides: glucose and galactose. Transgalactosylation results in the chain elongation and formation of GOS chains of different lengths. β (1→6) linked galactopyranosyl units linked to a terminal glucopyranosyl residue via an α(1→4) glycosidic bond.

These two steps occur simultaneously. Still, the relatively low efficiency of the transgalactosylation reaction and low COS yield represents a challenge for this method. This method results in a complex mixture containing glucose, galactose, high amounts of residual lactose, and GOS of different structures.

An object of this invention is to overcome the drawbacks of traditional methods of COS production, in particular, maximizing GOS yield and reducing the residual lactose level. However, the majority of research has been conducted on lactose-rich streams that are by-products of the cheese industry. These streams would typically contain salt, cultures or enzymes that can negatively impact the flavor profile, the purity, and further processibility of the stream.

Herein, the disclosed processes can start with a relatively concentrated lactose-containing product or stream, subject it to two consecutive enzymatic treatments, with heat treatment following each enzymatic step, and then a membrane filtration step. In the first enzymatic step, the lactose product is treated with a beta-galactosidase enzyme that converts lactose into GOS followed by heat deactivation of the enzyme after a high GOS yield is attained. The composition resulting from this step contains a mixture of GOS of different degrees of polymerization (DP), residual lactose, DP2, glucose, and galactose. In the second step, the composition is treated with a beta-galactosidase enzyme of a different source with higher hydrolytic activity compared to the enzyme in the first step. Additionally, this enzyme selectively hydrolyses lactose with minimal impact on OS. This step reduces residual lactose while maximizing the retention of GOS formed, and this step is also followed by heat deactivation. Lastly, membrane filtration is used for separating/concentrating to retain a higher purity GOS stream, while permeating glucose, galactose, DP2 sugars, and lactose. The amount of GOS in a composition herein is based on galactooligosaccharides having a degree of polymerization of three or more (DP3+).

### Processes for Producing Galactooligosaccharides

In accordance with an aspect of this invention, a process is provided for producing galactooligosaccharides (GOS), and in this aspect, the process can comprise (or consist essentially of, or consist of) (a) contacting a lactose-containing product and a first β-galactosidase enzyme to form a first composition, (b) deactivating the first β-galactosidase enzyme in the first composition, (c) adjusting a temperature of the first composition to within a range from 5 to 55° C., (d) contacting the first composition and a second β-galactosidase enzyme to form a second composition, (e) deactivating the second β-galactosidase enzyme in the second composition, and (f) subjecting the second composition to membrane filtration to form a concentrated galactooligosaccharides (GOS) composition.

Generally, the features of this process (e.g., the characteristics of the lactose-containing product, the first β-galactosidase enzyme, the characteristics of the first composition, the second β-galactosidase enzyme, the characteristics of the second composition, the characteristics of the GOS composition, and the conditions under which any of the steps are performed, among others) are independently described herein and these features can be combined in any combination to further describe the disclosed process. Moreover, other steps can be conducted before, during, and/or after any of the steps listed in the disclosed process, unless stated otherwise. Additionally, any GOS compositions (e.g., concentrated GOS compositions) produced in accordance with the disclosed process are within the scope of this disclosure and are encompassed herein.

Filtration technologies (e.g., ultrafiltration, nanofiltration, diafiltration, etc.) can separate or concentrate components in mixtures—such as milk—by passing the mixture through a membrane system (or selective barrier) under suitable conditions (e.g., pressure). The concentration/separation can be, therefore, based on molecular size. The stream that is retained by the membrane is called the retentate (or concentrate). The stream that passes through the pores of the membrane is called the permeate.

Referring now to step (a), a lactose-containing product is contacted with a first β-galactosidase enzyme to form a first composition. While not limited thereto, the lactose-containing product often can contain from 6 to 50 wt. % lactose, such as from 8 to 40 wt. %, from 10 to 30 wt. %, or from 10 to 20 wt. % lactose, and the like. In one aspect, for instance, the lactose-containing product can comprise a nanofiltration (NF) retentate fraction (typically, of an ultrafiltration permeate fraction of whole milk or skim milk). Optionally, the lactose-containing product can comprise a concentrated NF retentate fraction (e.g., in which the NF retentate fraction is concentrated via forward osmosis or reverse osmosis, or other suitable technique).

The whole milk prior to UF can be cow's milk, which contains approximately 87 wt. % water, 3-4 wt. % protein, 4-5 wt. % carbohydrates/lactose, 3-4 wt. % fat, and 0.3-0.8 wt. % minerals. Skim milk can be prepared by separating (e.g., centrifugally separating or microfiltering) the whole milk into skim milk and a fat-rich fraction (also referred toas cream or butterfat). The fat-rich fraction typically contains high levels of fat (e.g., 20-50 wt. % fat, 30-50 wt. %, 35-45 wt. % fat, or 38-42 wt. %) and solids (e.g., 30-60 wt. %, 40-55 wt. %, 40-50 wt. %, or 42-47 wt. %), aid often contains approximately 1-4 w t. % protein (or 1-3 wt. %, or 2-3 wt. %), 2-5 wt. % lactose (or 2.5-4 wt. %, or 2.5-3.5 wt. %), and 0.2-0.9 wt. % minerals (or 02-0.6 wt. %, or 0.2-0.4 wt. %), although not limited thereto. In contrast, the skim milk prior to UF typically contains very low levels of fat (e.g., less than or equal to 0.5 wt. %, less than or equal to 0.35 wt. %, or less than or equal to 0.2 wt. %) and much lower solids (e.g., 7-13 wt. %, 8-12 wt. %, 8.5-10 wt. %, or 9-9.5 wt. %) than the fat-rich fraction, and often the skim milk contains approximately 2-5 wt. % protein (or 3-4 wt. %, or 3.2-3.7 wt. %), 3-6 wt. % lactose (or 4-5.5 wt. %, or 4.5-5 wt. %), and 0.4-1.2 wt. % minerals (or 0.4-0.9 wt. %, or 0.5-0.9 wt. %), although not limited thereto.

In addition to the aforementioned lactose content, the lactose-containing product in step (a) can contain from 0.01 to 1 wt. %, from 0.01 to 0.5 wt. %, or from 0.01 to 0.2 wt. % fat, although not necessarily limited thereto. Additionally or alternatively, the lactose-containing product can contain from 0.1 to 2 wt. %, from 0.1 to 1.2 wt. %, or from 0.2 to 1 wt. % protein. Additionally or alternatively, the lactose-containing product can contain from 0.5 to 3 wt. %, from 0.7 to 2.5 wt. %, or from 1 to 2 wt. % minerals.

Any suitable β-galactosidase enzyme can be used as the first β-galactosidase enzyme in step (a). Generally, the first β-galactosidase enzyme is of the type that produces galactooligosaccharides (GOS) from lactose. In a particular aspect of step (a), the first β-galactosidase enzyme can be derived from Bifidobacterium bifidum.

Step (a) can be performed at any suitable temperature and time conditions. While not limited thereto, step (a) can be conducted at a first temperature in a range from 20 to 70° C., such as from 25 to 65° C. from 30 to 70° C., from 30 to 60° C., or from 35 to 55° C., and the like. In an aspect, step (a) can be performed at a temperature above ambient (e.g., 20-25° C.), thus the lactose-containing product and the first enzyme can be heated to a desired elevated temperature and contacted for a suitable period of time to form the first composition.

Referring now to the time period for step (a), often step (a) is conducted for a first time period that ranges from 15 min to 10 hr. Other illustrative ranges for the first time period include from 30 min to 8 hr, from 45 min to 6 hr, from 1 hr to 4 hr, or from 90 min to 150 min, and the like.

As one of skill in the art would readily recognize, the amount the first enzyme used in step (a) can vary significantly based on the first temperature, the first time period, and the amount of lactose in the lactose-containing product, among other considerations. While not being limited thereto, the amount of the first β-galactosidase enzyme can range from 0.05 to 1 wt. %, from 0.1 to 0.8 wt. %, or from 0.15 to 0.65 wt. %, based on a total weight of the lactose-containing product. Based on the starting amount of lactose, the amount of the first β-galactosidase enzyme can range from 1 to 15 wt. %, from 1.5 to 6 wt. %, from 1.5 to 4 wt. %, or from 2 to 3 wt. %, based on the amount of lactose in the lactose-containing product, although this amount can vary based on the temperature and time conditions in step (a), as referenced hereinabove.

Referring now to step (b), the first β-galactosidase enzyme in the first composition is deactivated. While any suitable method for deactivating the enzyme in step (b) can be utilized, deactivation is conveniently accomplished by exposure to a relatively high temperature for a suitable time. Thus, deactivating in step (b) can comprise subjecting the first composition to, or heat treating the first composition at, the elevated temperature.

Step (b) can be performed at any temperature and time conditions that sufficiently deactivate the first β-galactosidase enzyme in the first composition. While not limited thereto, deactivating in step (b) can comprise subjecting the first composition to, or heat treating the first composition at, a temperature in a range from 75 to 150° C., such as from 80 to 120° C., from 80 to 115° C., or from 85 to 105° C., and the like.

Referring now to the time period for step (b), deactivating in step (b) often comprises subjecting the first composition to, or heat treating the first composition at, the above temperature(s) for a time period that ranges from 1 min to 6 hr. Other illustrative ranges for the heat treatment time period to deactivate the enzyme in step (b) can include from t min to 30 min, from 2 min to 20 min, or from 4 min to 15 min, and the like.

In step (c), the temperature of the first composition is adjusted to within a range from 5 to 55° C. Hence, for instance, when high temperature enzyme deactivation is utilized in step (b), the first composition is cooled to a temperature within the range of 5 to 55° C. In one aspect, the temperature of the first composition in step (c) is from 10 to 55° C., while in another aspect, the temperature is from 20 to 50° C., and in yet another aspect, the temperature is from 25 to 45° C., and in still another aspect, the temperature is from 30 to 45° C. Other suitable temperatures and ranges can be used, if desired.

At this stage of the process (before step (d) and the addition of the second enzyme), the first composition often can contain from 1 to 5 wt. %, from 1.5 to 4.5 wt. %, or from 2 to 3.5 wt. % lactose, although not limited thereto. Additionally or alternatively, the first composition can contain from 4 to 10 wt. %, from 5 to 9 wt. %, or from 6 to 8.5 wt. % GOS. Any combination of the above lactose and GOS amounts can be present in the first composition, however, the weight ratio of GOS:lactose beneficially can range from 1.6:1 to 3.5:1, from 2:1 to 3:1, or from 2.2:1 to 2.8:1, although this ratio is not limited solely to these ranges.

Based on carbohydrates in the first composition, the first composition can contain from 10 to 20 wt. %, from 12 to 18 wt. %, or from 13 to 17 wt. % lactose. Additionally or alternatively, the first composition can contain from 30 to 44 wt. %, from 32 to 42 W. %, or from 34 to 40 wt. % GOS, based on carbohydrates.

After the first enzyme treatment, the first composition (before step (d)) contains significantly less lactose than is present in the lactose-containing product in step (a). Advantageously, the first composition contains from 60 to 95 wt. % less lactose than that which is present in the lactose-containing product in step (a). More often, the first composition contains from 70 to 90 wt. % or from 75 to 85 wt. % less lactose than that in the lactose-containing product in step (a).

In step (d), the first composition is contacted with a second β-galactosidase enzyme to form a second composition, and any suitable β-galactosidase enzyme can be used as the second β-galactosidase enzyme in step (d). Generally, the second β-galactosidase enzyme is of the type that preferentially hydrolyzes lactose to form glucose and galactose. In a particular aspect of step (d), the second β-galactosidase enzyme can be derived from Kluyveromyces lactis.

Step (d) can be performed at any suitable temperature and time conditions. While not limited thereto, step (d) can be conducted at a second temperature in a range from 10 to 60° C., such as from 20 to 50° C., from 25 to 45° C., or from 30 to 45° C., and the like. In an aspect, step (d) can be performed at a temperature above ambient (e.g., 20-25° C.), thus the first composition and the second enzyme can be heated to a (or controlled at a) desired elevated temperature and contacted for a suitable period of time to form the second composition.

Referring now to the time period for step (d), often step (d) is conducted for a second time period that ranges from 15 min to 10 hr. Other illustrative ranges for the second time period include from 30 min to 8 hr, from 45 min to 6 hr, from 1 hr to 4 hr, or from 90 min to 150 min, and the like.

As one of skill in the art would readily recognize, the amount the second enzyme used in step (d) can vary significantly based on the second temperature, the second time period, and the amount of lactose in the first composition, among other considerations. While not being limited thereto, the amount of the second β-galactosidase enzyme can range from 0.01 to 0.6 wt. %, from 0.03 to 0.3 wt. %, or from 0.04 to 0.1 wt. %, based on a total weight of the first composition. Based on the amount of lactose in the first composition, the amount of the second β-galactosidase enzyme can range from 0.35 to 20 wt. %, from 1 to 10 wt. %, or from 1.4 to 4 wt. %, based on the amount of lactose in the first composition, although this amount can vary based on the temperature and time conditions in step (d), as referenced hereinabove.

Referring now to step (e), the second β-galactosidase enzyme in the second composition is deactivated. While any suitable method for deactivating the enzyme in step (e) can be utilized, deactivation is conveniently accomplished by exposure to a relatively high temperature for a suitable time. Thus, deactivating in step (e) can comprise subjecting the second composition to, or heat treating the second composition at, the elevated temperature.

Step (e) can be performed at any temperature and time conditions that sufficiently deactivate the second β-galactosidase enzyme in the second composition. While not limited thereto, deactivating in step (e) can comprise subjecting the second composition to, or heat treating the second composition at, a temperature in a range from 75 to 150° C., from 80 to 120° C., from 80 to 115° C., or from 85 to 105° C., and the like.

Referring now to the time period for step (e), deactivating in step (e) often comprises subjecting the second composition to, or heat treating the second composition at, the above temperature(s) for a time period that ranges from 1 min to 6 hr. Other illustrative ranges for the heat treatment time period to deactivate the enzyme in step (e) can include from 1 min to 30 min, from 2 min to 20 min, or from 4 min to 15 min, and the like.

If heat treatment is used for deactivation in step (e), the process can further comprise a step of cooling the second composition to within a range from 5 to 50° C. (before step (f)). In one aspect, the temperature of the second composition can be cooled or adjusted to within a range from 10 to 45° C. or from 20 to 50° C., while in another aspect, the temperature of the second composition can be cooled or adjusted to within a range from 5 to 25° C. or from 8 to 20° C., prior to step (f). In an aspect, the temperature of the second composition prior to step (f) can be at or below ambient conditions (e.g., 20-25° C.), thus the second composition can be cooled to a temperature less than or equal to 25° C., less than or equal to 20° C., less than or equal to 18° C., or less than or equal to 15° C.; typical ranges include 8-18° C. and 10-15° C.

At this stage of the process (before step (f) and concentrating via membrane filtration), the second composition often can contain from 0.05 to 5 wt. %, from 0.05 to 1.5 wt. %, from 0.1 to 4 wt. %, from 0.1 to 1 wt. %, from 0.2 to 2 wt. %, or from 0.2 to 0.9 wt. % lactose, although not limited thereto. Additionally or alternatively, the second composition can contain from 4 to 15 wt. %, from 4 to 9 wt. %, from 5 to 12 wt. %, from 5 to 8 wt. %, from 5.5 to 9.5 wt. %, or from 5.5 to 7.5 wt. % GOS. Any combination of the above lactose and GOS amounts can be present in the second composition, however, the weight ratio of GOS:lactose in the second composition beneficially can range from 4:1 to 40:1, from 7:1 to 35:1, or from 8:1 to 30:1, although this ratio is not limited solely to these ranges.

Based on carbohydrates in the second composition, the second composition can contain from 0.5 to 5 wt. %, from 1 to 5 wt. %, or from 2 to 4 wt. % lactose. Additionally or alternatively, the second composition can contain from 20 to 45 wt. %, from 25 to 40 wt. %, or from 32 to 39 wt. % GOS, based on carbohydrates.

After the second enzyme treatment, the second composition (before step (f)) contains significantly less lactose than is present in the lactose-containing product in step (a). Advantageously, the second composition contains from 80 to 99 wt. % less lactose than that which is present in the lactose-containing product in step (a). More often, the second composition contains from 85 to 98 wt. %, from 90 to 99 wt. %, from 92 to 99 wt. % or from 93 to 98 wt. % less lactose than that in the lactose-containing product in step (a).

However, and beneficially, the amount of GOS present in the second composition is substantially the same as in the first composition. Ordinarily, before step (f), the second composition contains less than or equal to 3 wt. % less GOS than that present in the first composition. More often, the second composition contains less than or equal to 2 wt. %, less than or equal to 1 wt. %, less than or equal to 0.8 wt. %, or less than or equal to 0.5 wt. %, or less than or equal to 0.3 wt. %, less GOS than that in the first composition before step (d).

In step (f), the second composition is subjected to membrane filtration to form a concentrated galactooligosaccharides (GOS) composition. In an aspect, step (f) can comprise nanofiltering the second composition with a polymeric membrane (or alternatively, with a ceramic membrane) to form the concentrated GOS composition (also can be referred to as the NF retentate). In another aspect, step (f) can comprise diafiltering the second composition with a nanofiltration membrane (polymeric or ceramic). While not wishing to be bound by the following theory, it is believed that polymeric membrane filtration is generally more suitable than ceramic membrane filtration in step (f) due to the need for smaller pore sizes and a low molecular weight cutoff. Further, diafiltering the second composition with the nanofiltration membrane can comprise diafiltering a mixture of the second composition and water. Any suitable water source can be used in the mixture, such as RO permeate.

Nanofiltration (or diafiltration) can be conducted using nanofiltration membranes with pore sizes that typically are in the 0.001 to 0.01 micron range, for example, pore sizes in a range from 0.001 to 0.008 μm. In some aspects, the step of nanofiltration utilizes a membrane system having pore sizes in a range from 0.001 to 0.01 μm.

Nanofiltration in the dairy industry typically uses membrane elements that retain particles with molecular weights above a certain molecular weight (measured in Daltons, Da). Nanofiltration is a pressure driven process in which the starting composition is forced through the membrane under pressure, and materials having a molecular weight greater than the specified cut-off generally are retained, while smaller particles generally pass though the membrane pores. Accordingly, nanofiltration can simultaneously perform both concentration and separation.

Referring to the membrane filtration in step (f), the membranes often can be identified based on molecular weight cut-off (MWCO), rather than pore size. The membrane system, such as a polymeric nanofiltration system, generally retains materials having molecular weights greater than the MWCO number(s). The second composition can be subjected to membrane filtration utilizing a membrane having a molecular weight cut-off (MWCO) at least 150 Da and less than 1000 Da. Therefore, suitable membranes can have a MWCO in a range from 150 Da to 900 Da; alternatively, from 150 Da to 500 Da; alternatively, from 150 to 300 Da; alternatively, from 200 to 900 Da; alternatively, from 200 to 800 Da; alternatively, from 300 to 900 Da; alternatively, from 300 to 800 Da; alternatively, from 300 to 700 Da; alternatively, from 300 to 500 Da; alternatively, from 500 to 1000 Da; alternatively, from 500 to 800 Da; alternatively, from 500 to 700 Da: alternatively, from 600 to 900 Da; or alternatively, from 600 to 800 Da. As an example, subjecting the second composition to membrane filtration utilizing a membrane having a molecular weight cut-off (MWCO) in a range from 300 to 800 Da encompasses the use of a membrane having a 300-500 Da MWCO, and the use of a membrane having a 500-700 Da MWCO, and the use of a membrane having a 600-800 Da MWCO.

In one aspect, the MWCO of the membrane utilized in step (f) is in a range from 200 to 800 Da, while in another aspect, the MWCO is from 300 to 800 Da, and in another aspect, the MWCO is from 300 to 700 Da, and in yet another aspect, the MWCO is from 500 to 800 Da, and in still another aspect, the MWCO is from 500 to 700 Da.

Step (f) can be conducted at a filtration temperature in a range from 5 to 50° C., although not limited thereto. In one aspect, the filtration temperature in step (f) can range from 10 to 45° C. or from 20 to 50° C. while in another aspect, the filtration temperature in step (f) can range from 5 to 25° C. or from 8 to 20° C. In an aspect, the filtration temperature in step (f) can be at or below ambient conditions (e.g., 20-25° C.), thus the filtration temperature can be less than or equal to 25° C., less than or equal to 20° C., less than or equal to 18° C., or less than or equal to 15° C.; typical ranges include 8-18° C. and 10-15° C.

Operating pressures for membrane filtration step (f) are not particularly limited, but generally range from 100 to 1000 psig. In one aspect, step (f) is conducted at a pressure in a range from 200 to 800 psig, while in another aspect, the pressure is in a range from 300 to 650 psig, and in yet another aspect, the pressure is in a range from 320 to 560 psig.

At this stage of the process (after step (f) and concentrating/separating via membrane filtration), the concentrated GOS composition often can contain from 0.1 to 3 wt. %, from 0.5 to 2 wt. %, or from 0.7 to 1.8 wt. % lactose, although not limited thereto. Additionally or alternatively, the concentrated GOS composition can contain from 10 to 20 wt. %, from 12 to 20 wt. %, or from 14 to 18 wt. % GOS. Any combination of the above lactose and GOS amounts can be present in the concentrated GOS composition, however, the weight ratio of GOS:lactose in the concentrated GOS composition beneficially can range from 6:1 to 45:1, from 7:1 to 35:1, or from 8:1 to 30:1, although this ratio is not limited solely to these ranges.

Based on carbohydrates in the concentrated GOS composition, the concentrated GOS composition can contain from 0.5 to 6 wt. %, from 1 to 5.5 wt. %, from 2 to 6 wt. %, or from 2.5 to 5 wt. % lactose. Additionally or alternatively, the concentrated GOS composition can contain from 24 to 50 wt. %, from 30 to 48 wt. %, from 33 to 50 wt. %, or from 38 to 46 wt. % GOS, based on carbohydrates.

Optionally, after step (f), the concentrated GOS composition can be heat treated. In one aspect, the step of heat treating can comprise pasteurizing at a temperature in a range from 80° C. to 95° C. for a time period m a range from less than one min up to 15 min, such as from 2 to 15 min. In another aspect, the step of heat treating can comprise UHT sterilization at a temperature in a range from 135° C. to 145° C. for a time period in a range from 1 to 10 sec. In yet another aspect, the step of heat treating can comprise UHT sterilization at a temperature in a range from 148° C. to 165 for a time period in a range from 0.05 to 1 sec, for example, from 150° C. to 155° C. for a time period in a range from 0.08 to 0.2 sec. Other appropriate pasteurization or sterilization temperature and time conditions are readily apparent from this disclosure. Further, this invention is not limited by the method or equipment used for performing the pasteurization/sterilization process—any suitable technique and apparatus can be employed, whether operated batchwise or continuously.

Typical UHT sterilization techniques include indirect heating, direct steam injection, direct steam infusion, and the like. For indirect heating, the GOS composition is not contacted directly with the heat source or heating medium, e.g., like a heat exchanger. Due to the heat transfer limitations, indirect heating requires a longer time for sterilization. Beneficially, in aspects of this invention, the GOS composition is heat treated using direct UHT sterilization. In direct steam injection, high temperature steam is injected into the pipe or other vessel containing the GOS composition, thus rapidly sterilizing the GOS composition. Direct steam injection generally is performed continuously—a continuous flow of the GOS composition is combined with a continuous injection of steam. In direct steam infusion, the GOS composition is sprayed into a chamber containing steam, thus rapidly and uniformly sterilizing the GOS composition. Like direct steam injection, direct steam infusion generally is performed continuously. After the heat treatment step, the heat treated GOS composition can be cooled to any suitable temperature, such as in a range from 5° C. to 40° C., or from 10° C. to 30° C.

The processes for producing galactooligosaccharides described herein can be performed batchwise or continuously. The examples that follow are batch experiments, but any step in the process, or any combination of steps, alternatively can be performed continuously. Any suitable vessel (e.g., a tank, a silo, etc.) can be used to perform any step in the process, any combination of steps in the process, or all steps in the process. The processes also can further comprise a step of packaging (aseptically or otherwise) the GOS composition in any suitable container and under any suitable conditions, and illustrative and non-limiting examples of typical containers include a cup, a bottle, a bag, or a pouch, and the like. The container can be made from any suitable material, such as glass, metal, plastics, and the like, as well as combinations thereof.

In some aspects, the process for producing galactooligosaccharides can further comprise the steps of (i) determining an amount of lactose, an amount of GOS, a ratio of GOS:lactose, or any combination thereof, in the first composition before step (d), and (ii) adjusting the first temperature, the first time period, the amount of the first β-galactosidase enzyme, or any combination thereof, in step (a) based on the determined composition feature of the first composition. For instance, if the amount of lactose in the first composition is too high, the first temperature can be adjusted, the first time period can be adjusted, the amount of the first β-galactosidase enzyme can be adjusted, or any combination of these can be adjusted, so that the amount of lactose in the first composition is reduced (e.g., to a desired target amount).

Likewise, the process for producing galactooligosaccharides can further comprise the steps of (i) determining an amount of lactose, an amount of GOS, a ratio of GOS:lactose, or any combination thereof, in the second composition before step (f), and (ii) adjusting the second temperature, the second time period, the amount of the second β-galactosidase enzyme, or any combination thereof, in step (d) based on the determined composition feature of the second composition. For instance, if the amount of lactose in the second composition is too high, the second temperature can be adjusted, the second time period can be adjusted, the amount of the second β-galactosidase enzyme can be adjusted, or any combination of these can be adjusted, so that the amount of lactose in the second composition is reduced (e.g., to a desired target amount).

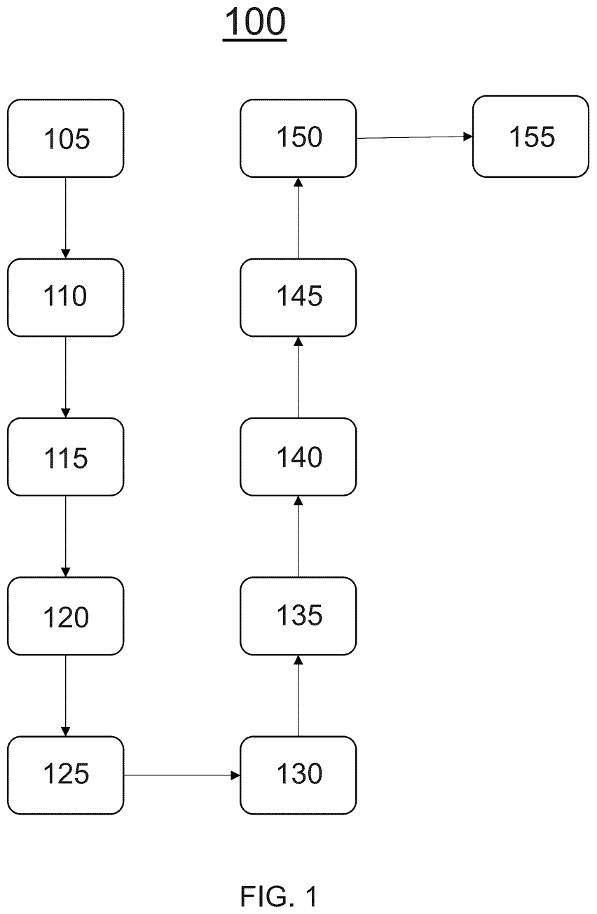

An illustrative and non-limiting example of a representative process 100 for producing galactooligosaccharides (GOS) consistent with aspects of this invention is shown in FIG. 1. First, a lactose-containing product 105, which in FIG. 1 contains 10-20 wt. % lactose, is heated 110 to a temperature in the 35-55° C. range, and then contacted 115 with a first β-galactosidase enzyme that can be derived from Bifidobacterium bifidum, followed by incubating 120 at a suitable combination of temperature and time, such as 35-55° C. for 90 to 150 min. The resulting first composition is then heat treated 125 at 85-105° C. for 5 to 10 min to deactivate the first β-galactosidase enzyme. After cooling 130 to a temperature of 30-45° C., a second β-galactosidase enzyme, which can be derived from Kluyveromyces lactis, is added 135, followed by incubating 140 at a suitable combination of temperature and time, such as 30-45° C. for 90 to 150 min. The resulting second composition is then heat treated 145 at 85-105° C. for 5 to 10 min to deactivate the second β-galactosidase enzyme, followed by membrane filtering 150 to form a retentate fraction, which is a concentrated galactooligosaccharides (GOS) composition 155.

### Galactooligosaccharides (GOS) Compositions

An illustrative and non-limiting example of a galactooligosaccharides (GOS) composition consistent with the present invention can contain from 0.5 to 6 wt. % lactose and from 24 to 50 wt. % GOS (DP3+), based on carbohydrates. Another illustrative and non-limiting example of a GOS composition consistent with the present invention contain lactose and GOS at a weight ratio of GOS:lactose that ranges from 6:1 to 45:1. Yet another illustrative and non-limiting example of a GOS composition consistent with the present invention can contain from 0.5 to 6 wt. % lactose and from 24 to 50 wt. % GOS, based on carbohydrates, and wherein the weight ratio of GOS:lactose in the composition ranges from 6:1 to 45:1. These illustrative and non-limiting examples of GOS compositions consistent with the present invention also can have any of the features listed below and in any combination, unless indicated otherwise.

In an aspect, any GOS composition described herein can contain from 1 to 5.5 wt. % lactose, or from 30 to 48 wt. % GOS, or both 1 to 5.5 wt. % lactose and 30 to 48 wt. % GOS. In another aspect, the GOS composition can contain from 2 to 6 wt. % lactose, or from 33 to 50% wt. % GOS, or both 2 to 6 wt. % lactose and 33 to 50 wt. % GOS. Additionally or alternatively, the GOS composition can contain from 2.5 to 5 wt. % lactose, or from 38 to 46 wt. % GOS, or both 2.5 to 5 wt. % lactose and 38 to 46 wt. % GOS. The respective amounts of lactose and GOS in the composition are based on total carbohydrates. Additionally or alternatively, the weight the ratio of GOS:lactose in the GOS composition can fall within a range from 7:1 to 35:1, from 7:1 to 15:1, from 8:1 to 30:1, or from 8:1 to 12:1.

As one of skill in the art would readily recognize, the GOS composition is not solely limited to lactose and GOS components. For instance, the GOS composition can further comprise glucose, galactose, and DP2 carbohydrates; alternatively, glucose and galactose; alternatively, glucose; alternatively, galactose; or alternatively, DP2 carbohydrates. In an aspect, the amount of galactose (or glucose) present in the GOS composition is greater than that of the lactose. For instance, the weight ratio of galactose:lactose in the GOS composition can fall within a range from 1.5:1 to 35:1, from 1.7:1 to 10:1, from 1.7:1 to 3:1, from 2:1 to 301, from 2:1 to 10:1, or from 2:1 to 3.1. The weight ratio of glucose:lactose in the GOS composition often can be higher, with typical ranges including from 3:1 to 40:1, from 3:1 to 20:1, from 3:1 to 7:1, from 4:1 to 40:1, from 4:1 to 10:1, or from 4:1 to 6:1. While not limited thereto, any GOS composition described herein can contain from 5 to 20 wt. % galactose, such as from 6 to 15 wt. % or from 7 to 10 wt. % galactose, and/or from 15 to 30 wt. % glucose, such as from 18 to 26 wt. % or from 20 to 24 wt. % glucose. These amounts are based on total carbohydrates.

Likewise, the GOS composition is not limited to carbohydrates, and also can contain fat, protein, and minerals. While not limited thereto, the GOS composition can contain, for instance, from 0.01 to 2 wt. % fat, and more often, from 0.03 to 1 wt. % or from 0.03 to 0.2 wt. % fat. Additionally or alternatively, the GOS composition can contain from 0.3 to 3 wt. % protein, and more often, from 0.4 to 1.5 wt. % or from 0.6 to 1.2 wt. % protein. Additionally or alternatively, the GOS composition can contain from 0.5 to 5 wt. % minerals, and more often, from 1 to 3 wt. % or from 1.5 to 2.5 wt. % minerals.

### EXAMPLES

The invention is further illustrated by the following examples, which are not to be construed in any way as imposing limitations to the scope of this invention. Various other aspects, modifications, and equivalents thereof which, after reading the description herein, can suggest themselves to one of ordinary skill in the art without departing from the spirit of the present invention or the scope of the appended claims.

Total solids (wt. %) were determined in accordance with procedure SMEDP 15.10 C by CEM Turbo Solids and Moisture Analyzer (CEM Corporation, Matthews, North Carolina). Ash is the residue remaining after ignition in a suitable apparatus at 550° C. to a constant weight; such treatment at 550° C. typically eliminates all organic matter, with the remaining material being primarily minerals (Standard Methods for the examination of dairy products, 17^{th} edition (2004), American Public Health Association, Washington DC). The ash test was performed by using a Phoenix (CEM Microwave Furnace), which heated the samples at 550° C. for 30 min. The mineral content (in wt. %) is generally similar to the ash content (wt. %), and thus the result of an ash test is used for quantification of the total mineral content in this disclosure. Protein content and fat content were determined by AOAC (Association of Official Analytical Chemists) methods.

Carbohydrate profiles (glucose, galactose, lactose, DP2, DP3, DP4 and DP4+) were determined by High Performance Liquid Chromatography (HPLC) using different detectors. HPLC-RI (refractive index) was employed to quantify galactose, glucose, DP2, DP3, DP4, and DP4+. HPAEC-PAD (Pulsed amperometric detection) method was used to separate DP2 GOS from lactose.

### Example 1

FIG. 1 summarizes the process used in Example 1, and Table I summarizes the carbohydrate breakdown of certain starting, intermediate, and final compositions. The starting lactose-containing product used in the examples had 17-22 wt. % solids and a pH of 5.8-7.0 and contained 0.01-0.10 wt. % fat, 0.2-0.8 wt. % protein, 1-2 wt. % minerals, and 15-20 wt. % carbohydrates (the balance being water).

In the first enzymatic step, each liter of lactose-containing product was combined with 3.6 g of a beta-galactosidase enzyme (originating from Bifidobacterium bifidum) in a stirred lank to form a first composition at a temperature of 55° C. for 120 min to maximize the GOS yield. This first composition was gently mixed throughout in order to prevent settling of the lactose and to increase the interaction between the enzyme and the building blocks of GOS within the tank. Enzyme deactivation was then performed at 95° C. for 7.5 min to minimize or stop the hydrolytic action of the enzyme. If the deactivation is not done at the appropriate time, the reaction equilibrium can shift towards hydrolyzing GOS, thereby reducing GOS yield.

After the first enzymatic step, the temperature was reduced to 37° C., and the first composition contained a mixture of GOS of different degrees of polymerization (DP), residual lactose, DP2, glucose, and galactose (see Table I). In the second enzymatic step, the first composition was contacted with approximately 0.06 wt. % of a different beta-galactosidase enzyme (originating from Kluyveromyces lactis) to form a second composition at a temperature of 37° C. for 120 min, and this step reduced the amount of residual lactose by hydrolyzing it into glucose and galactose. The enzyme is this step has a higher hydrolytic activity and is highly selective towards breaking down lactose, with lower affinity towards GOS. Gentle mixing was maintained throughout this step to increase the interaction between second enzyme and the residual lactose. The temperature and time utilized for the second enzymatic treatment step generally is selected to achieve significant lactose reduction (e.g., 65-85%) while maintaining almost all of the GOS (e.g., 95% or more).

Deactivation of the second enzyme was performed at 95° C. for 7.5 min to minimize or stop enzymatic cleavage of GOS. If the deactivation is not done at the appropriate time, the GOS yield will be reduced. After the second enzymatic step, the temperature was reduced to 5-10° C., and the second composition contained a mixture of GOS of different degrees of polymerization (DP), residual lactose, DP2, glucose, and galactose (see Table I).

The second composition was subjected to membrane filtration at a temperature ranging between 10-20° C. and a pressure ranging between 360-560 psig to concentrate approximately two-fold the (high molecular weight) GOS while permeating other (lower molecular weight) sugars including DP2, lactose, glucose, and galactose. A nanofiltration unit was used to form the GOS composition (retentate) and NF permeate. The nanofiltration unit employed polymeric membrane filters having a MWCO of 500-700 daltons.

Beneficially, in the process of Example 1 as shown in Table I, the first composition contained 6.8 wt. % GOS and only 2.8 wt. % lactose (weight ratio of GOS:lactose equal to 2.4:1). On a carbohydrate basis, the first composition contained 36 wt. % GOS and only 15 wt. % lactose, and the first composition contained 80 wt. % less lactose than that present in the starting lactose-containing product.

Table I demonstrates the surprisingly high amount of GOS to lactose in the second composition. The second composition contained 6.5 wt. % GOS (only 0.2-0.3 wt. % less than in the first composition) and only 0.7 wt. % lactose (weight ratio of GOS:lactose equal to 9.3:1). On a carbohydrate basis, the second composition contained 35 wt. % GOS and only 3.8 wt. % lactose, and the second composition contained 95 wt. % less lactose than that present in the starting lactose-containing product.

After the membrane filtration step, the concentrated GOS composition unexpectedly had a higher relative amount of GOS to lactose, while the overall amounts of carbohydrates were increased (higher % solids). The GOS composition contained 15.6 wt. % GOS (DP3+) and only 1.5 wt. % lactose (weight ratio of GOS:lactose equal to 10.3:1). On a carbohydrate basis, the GOS composition contained 43 wt. % GOS and only 4 wt. % lactose. The GOS composition in Table I had 35-36 wt. % solids and contained approximately 0.09 wt. % fat, 0.95 wt. % protein, and 2.1 wt. % minerals.

TABLE I
Carbohydrate profiles for Example 1 (values in wt. %)

| | | | | | | DP4 | GOS/ | |
|---|---|---|---|---|---|---|---|---|
| | | Glu- | Galac- | Lac- | | and | Lac- | Total |
| | DP2 | cose | tose | tose | DP3 | DP4+ | tose | GOS |
| Starting | 2.14 | 0 | 0.07 | 14.43 | 0.03 | 0.31 | 0.02:1 | 0.34 |
| lactose- | | | | | | | | |
| containing | | | | | | | | |
| product | | | | | | | | |
| (19-20 wt. | | | | | | | | |
| % solids) | | | | | | | | |
| First | 3.66 | 4.43 | 1.03 | 2.83 | 4.31 | 2.46 | 2.4:1 | 6.77 |
| compo- | | | | | | | | |
| sition | | | | | | | | |
| (after | | | | | | | | |
| first | | | | | | | | |
| enzymatic | | | | | | | | |
| step, | | | | | | | | |
| 19-20 wt. | | | | | | | | |
| % solids) | | | | | | | | |
| Second | 3.36 | 5.69 | 2.25 | 0.70 | 4.09 | 2.44 | 9.3:1 | 6.53 |
| compo- | | | | | | | | |
| sition | | | | | | | | |
| (after | | | | | | | | |
| second | | | | | | | | |
| enzymatic | | | | | | | | |
| step, | | | | | | | | |
| 19-20 wt. | | | | | | | | |
| % solids) | | | | | | | | |
| GOS | 7.86 | 7.91 | 3.26 | 1.52 | 9.93 | 5.72 | 10.3:1 | 15.65 |
| compo- | | | | | | | | |
| sition | | | | | | | | |
| (after | | | | | | | | |
| membrane | | | | | | | | |
| filtration, | | | | | | | | |
| 35-36 wt. | | | | | | | | |
| % solids) | | | | | | | | |

### Examples 2-4

In Examples 2-4, a representative first composition having 18-21 wt. % total solids, with the components and amounts shown in Table II, was treated with different amounts of the second enzyme (0.06 wt. %, 0.1 wt. %, 0.4 wt. %) and incubated for different times (90 min, 120 min, 180 min) at a fixed temperature of 37° C. to determine the impact on lactose, GOS (DP3+), and the weight ratio of GOS:lactose in the second composition (total solids were in the 18-21 wt. % range). Table II summarizes the results.

Higher enzyme loadings reduced both lactose and GOS amounts in the second composition, and generally increased the GOS:lactose ratio. However, surprisingly high GOS:lactose ratios often accompanied a significantly lower amount of GOS in the second composition (e.g., a lower GOS yield). Longer incubation time periods also generally reduced both lactose and GOS amounts in the second composition, and generally increased the GOS:lactose ratio.

TABLE II
Carbohydrate profiles for Examples 2-4 (values in wt. %)

| | | | | | | | | |
|---|---|---|---|---|---|---|---|---|
| Time | | | | | | DP4 and | GOS/ | Total |
| (min) | DP2 | Glucose | Galactose | Lactose | DP3 | DP4+ | lactose | GOS |

First composition (after first enzymatic step)

| | | | | | | | | |
|---|---|---|---|---|---|---|---|---|
| — | 3.30 | 4.63 | 1.03 | 2.94 | 4.69 | 3.04 | 2.6:1 | 7.73 |

Example 2 - Second composition (after second enzymatic step) - 0.06 wt. %

| | | | | | | | | |
|---|---|---|---|---|---|---|---|---|
| 90 | 2.34 | 6.22 | 2.58 | 0.95 | 4.41 | 3.13 | 7.9:1 | 7.55 |
| 120 | 2.64 | 6.46 | 2.90 | 0.52 | 4.29 | 3.13 | 14.3:1 | 7.42 |
| 180 | 2.05 | 6.76 | 3.36 | 0.37 | 4.09 | 3.10 | 19.6:1 | 7.19 |

Example 3 - Second composition (after second enzymatic step) - 0.1 wt. %

| | | | | | | | | |
|---|---|---|---|---|---|---|---|---|
| 90 | 1.92 | 6.61 | 3.17 | 0.63 | 4.13 | 3.09 | 11.4:1 | 7.21 |
| 120 | 2.00 | 6.82 | 3.52 | 0.34 | 3.96 | 3.06 | 20.5:1 | 7.01 |
| 180 | 1.44 | 7.09 | 4.03 | 0.28 | 3.69 | 2.99 | 23.5:1 | 6.68 |

Example 4 - Second composition (after second enzymatic step) - 0.4 wt. %

| | | | | | | | | |
|---|---|---|---|---|---|---|---|---|
| 90 | 0.87 | 7.37 | 4.83 | 0.18 | 3.09 | 2.75 | 32.4:1 | 5.84 |
| 120 | 0.61 | 7.53 | 5.19 | 0.16 | 2.84 | 2.67 | 34.4:1 | 5.51 |
| 180 | 0.47 | 7.72 | 5.63 | 0.14 | 2.47 | 2.56 | 35.9:1 | 5.03 |

### Examples 5-9

Examples 5-9 were conducted to determine the impact of the molecular weight cut-off (MWCO) of the polymeric nanofiltration membrane on the distribution of carbohydrates (sugar and GOS) in the NF retentate and NF permeate. In Example 5 (MWCO of 200 Da) and Example 8 (MWCO of 600-800 Da), a representative first composition before filtration (18.7 wt. % solids, 0.05 wt. % fat, 0.66 wt. % protein, and 1.15 wt. % minerals) with the components and amounts shown in Table III, was subjected to membrane filtration at a temperature ranging between 5-50° C. and a pressure ranging between 200-500 psig. In Example 6 (MWCO of 150-300 Da) and Example 7 (MWCO of 300-500 Da), a representative first composition before filtration (21.2 wt. % solids, 0.05 wt. % fat, 0.66 wt. % protein, and 1.22 wt. % minerals) with the components and amounts shown in Table I, was subjected to membrane filtration at a temperature ranging between 5-45° C. and a pressure ranging between 350-550 psig.

The NF permeates of Examples 5-7 contained very little GOS and thus the NF retentates contained almost all of the GOS present in the composition before filtration, i.e., very good GOS yield. However, these NF permeates also did not contain large amounts of the sugars (DP2, glucose, galactose, lactose), indicating that much of the sugars also were in the NF retentates, i.e., both sugar and GOS were concentrated. These MWCO's therefore are useful where GOS yield is very important but significant concentration of GOS relative to sugar is not required (note the GOS/sugar ratio in the NF retentate).

The NF permeate of Example 8 contained a significant amount of GOS and the GOS/sugar ratio in the NF retentate was very high. The MWCO of Example 8 permeated a fair amount of GOS, thus GOS yield in the NF retentate was lower, but significant concentration of GOS relative to sugar was observed in the NF retentate.

Example 9 (MWCO of 500-700 Da) was performed with a different starting material, and the representative second composition before filtration (19.3 wt. % solids, 0.06 wt. % fat, 0.4 wt. % protein, and 1.07 wt. % minerals) with the components and amounts shown in Table III, was subjected to membrane filtration at a temperature ranging between 10-20° C. and a pressure ranging between 320-560 psig. This example had a good balance between GOS yield in the NF retentate (relatively low GOS in the NF permeate) and concentrating GOS relative to sugar in the NF retentate (GOS/ratio of the NF retentate versus the composition before filtration). The NF retentate (GOS composition) of Example 9 in Table III had 35-36 wt. % solids and contained approximately 0.05 wt. % fat, 1 wt. % protein, and 2 wt. % minerals.

TABLE III
Carbohydrate profiles for Examples 5-9 (values in wt. %)

| | | | | | | | | |
|---|---|---|---|---|---|---|---|---|
| | | | | | DP4/ | Total | Total | GOS/ |
| DP2 | Glucose | Galactose | Lactose | DP 3 | DP4+ | GOS | sugar | sugar |

Example 5 - NF with 200 Da MWCO

| | | | | | | | | | |
|---|---|---|---|---|---|---|---|---|---|
| Before filtration | 3.63 | 3.94 | 0.96 | 2.68 | 4.10 | 2.44 | 6.55 | 11.21 | 0.58 |
| NF permeate | 0.09 | 2.03 | 0.46 | 0.05 | 0.01 | 0.00 | 0.01 | 2.64 | <0.01 |
| NF retentate | 3.96 | 4.40 | 1.09 | 3.86 | 5.10 | 2.96 | 8.05 | 13.30 | 0.61 |

Example 6 - NF with 150-300 Da MWCO

| | | | | | | | | | |
|---|---|---|---|---|---|---|---|---|---|
| Before filtration | 6.96* | 4.58 | 1.13 | — | 4.86 | 2.88 | 7.74 | 12.67 | 0.61 |
| NF permeate | 0.09* | 0.57 | 0.13 | — | 0.03 | 0.22 | 0.25 | 0.79 | 0.32 |
| NF retentate | 11.70* | 7.00 | 1.76 | — | 8.04 | 4.61 | 12.65 | 20.47 | 0.62 |

Example 7 - NF with 300-500 Da MWCO

| | | | | | | | | | |
|---|---|---|---|---|---|---|---|---|---|
| Before filtration | 6.96* | 4.58 | 1.13 | — | 4.86 | 2.88 | 7.74 | 12.67 | 0.61 |
| NF permeate | 0.23* | 2.14 | 0.48 | — | 0.04 | 0.23 | 0.27 | 2.85 | 0.09 |
| NF retentate | 12.61* | 6.51 | 1.70 | — | 9.11 | 5.21 | 14.32 | 20.83 | 0.69 |

Example 8 - NF with 600-800 Da MWCO

| | | | | | | | | | |
|---|---|---|---|---|---|---|---|---|---|
| Before filtration | 3.63 | 3.94 | 0.96 | 2.68 | 4.10 | 2.44 | 6.55 | 11.21 | 0.58 |
| NF permeate | 2.53 | 3.78 | 0.92 | 2.92 | 2.99 | 1.13 | 4.12 | 10.16 | 0.41 |
| NF retentate | 5.82 | 3.75 | 0.95 | 2.94 | 8.81 | 7.76 | 16.57 | 13.46 | 1.23 |

Example 9 - NF with 500-700 Da MWCO

| | | | | | | | | | |
|---|---|---|---|---|---|---|---|---|---|
| Before filtration | 3.32 | 5.83 | 2.34 | 0.55 | 4.07 | 2.47 | 6.54 | 12.04 | 0.54 |
| NF permeate | 0.26 | 3.94 | 1.51 | 0.05 | 0.09 | 0.23 | 0.32 | 5.75 | 0.06 |
| NF retentate | 7.62 | 7.79 | 3.15 | 1.50 | 9.76 | 5.60 | 15.36 | 20.06 | 0.77 |

*Lactose was not measured separately from DP2. These numbers represent total DP2 carbohydrates.

1. A process comprising:
(a) contacting a lactose-containing product and a first β-galactosidase enzyme to form a first composition;
(b) deactivating the first β-galactosidase enzyme in the first composition;
(c) adjusting a temperature of the first composition to within a range from 5 to 55° C.;
(d) contacting the first composition and a second β-galactosidase enzyme to form a second composition;
(e) deactivating the second β-galactosidase enzyme in the second composition; and
(f) subjecting the second composition to membrane filtration to form a concentrated galactooligosaccharides (GOS) composition.

2-30. (canceled)

31. The process of claim 1, wherein the lactose-containing product comprises:
from 6 to 50 wt. % lactose;
from 0.01 to 1 wt. % fat;
from 0.1 to 2 wt. % protein;
from 0.5 to 3 wt. % minerals; or
any combination thereof.

32. The process of claim 1, wherein the lactose-containing product comprises a nanofiltration (NF) retentate fraction of an ultrafiltration (UF) permeate fraction of whole milk or skim milk.

33. The process of claim 1, wherein:
an amount of the first β-galactosidase enzyme is from 1 to 15 wt. %, based on an amount of lactose in the lactose-containing product;
an amount of the first β-galactosidase enzyme is from 0.05 to 1 wt. %, based on a total weight of the lactose-containing product;
the first β-galactosidase enzyme is derived from Bifidobacterium bifidum;
step (a) is conducted at a first temperature in a range from 20 to 70° C.;
step (a) is conducted for a first time period in a range from 15 min to 10 hr; or
any combination thereof.

34. The process of claim 1, wherein:
deactivating in step (b) comprises subjecting the first composition to, or heat treating the first composition at, a temperature in a range from 75 to 150° C.;
deactivating in step (b) comprises subjecting the first composition to, or heat treating the first composition at, the temperature for a time period in a range from 1 min to 6 hr; and
the temperature of the first composition in step (c) is from 10 to 55° C.

35. The process of claim 1, wherein before step (d), the first composition contains:
from 1 to 5 wt. % lactose;
from 4 to 10 wt. % GOS;
a weight ratio of GOS:lactose from 1.6:1 to 3.5:1;
from 10 to 20 wt. % lactose, based on carbohydrates;
from 30 to 44 wt. % GOS, based on carbohydrates;
from 60 to 95 wt. % less lactose than in the lactose-containing product in step (a); or
any combination thereof.

36. The process of claim 1, wherein:
an amount of the second β-galactosidase enzyme is from 0.01 to 0.6 wt. %, based on a total weight of the first composition;
an amount of the second β-galactosidase enzyme is from 0.35 to 20 wt. %, based on an amount of lactose in the first composition;
the second β-galactosidase enzyme is derived from Kluyveromyces lactis;
step (d) is conducted at a second temperature in a range from 10 to 60° C.;
step (d) is conducted for a second time period in a range from 15 min to 10 hr; or
any combination thereof.

37. The process of claim 1, wherein:
deactivating in step (e) comprises subjecting the second composition to, or heat treating the second composition at, a temperature in a range from 75 to 150° C.;
deactivating in step (e) comprises subjecting the second composition to, or heat treating the second composition at, the temperature for a time period in a range from 1 min to 6 hr; and
the process further comprises a step of cooling the second composition to a temperature within a range from 5 to 50° C. prior to step (f).

38. The process of claim 1, wherein before step (f), the second composition contains:
from 0.05 to 5 wt. % lactose;
from 4 to 15 wt. % GOS;
a weight ratio of GOS:lactose from 4:1 to 40:1;
from 0.5 to 5 wt. % lactose, based on carbohydrates;
from 20 to 45 wt. % GOS, based on carbohydrates;
from 80 to 99 wt. % less lactose than that in the lactose-containing product in step (a);
less than or equal to 3 wt. % less GOS than in the first composition; or
any combination thereof.

39. The process of claim 1, wherein:
step (f) comprises nanofiltering the second composition with a polymeric membrane;
step (f) comprises diafiltering the second composition with a nanofiltration membrane;
step (f) is conducted at a filtration temperature in a range from 5 to 50° C.;
step (f) is conducted at a pressure in a range from 100 to 1000 psig;
the process further comprises heat treating the GOS composition after step (f); or
any combination thereof.

40. The process of claim 1, wherein the second composition is subjected to the membrane filtration utilizing a membrane having a molecular weight cut-off (MWCO) in a range from 300 Da to 800 Da.

41. The process of claim 1, wherein the concentrated GOS composition contains:
from 0.1 to 3 wt. % lactose;
from 10 to 20 wt. % GOS;
a weight ratio of GOS:lactose from 6:1 to 45:1;
from 0.5 to 6 wt. % lactose, based on carbohydrates;
from 24 to 50 wt. % GOS, based on carbohydrates; or
any combination thereof.

42. The process of claim 1, further comprising the steps of:
(i) determining an amount of lactose, an amount of GOS, a ratio of GOS:lactose, or any combination thereof, in the first composition before step (d); and
(ii) adjusting a first temperature, a first time period, an amount of the first β-galactosidase enzyme, or any combination thereof, in step (a) based on a determined composition feature of the first composition in step (i).

43. The process of claim 1, further comprising the steps of:
(i) determining an amount of lactose, an amount of GOS, a ratio of GOS:lactose, or any combination thereof, in the second composition before step (f); and
(ii) adjusting a second temperature, a second time period, an amount of the second β-galactosidase enzyme, or any combination thereof, in step (d) based on a determined composition feature of the second composition in step (i).

44. A composition comprising:
from 0.5 to 6 wt. % lactose and from 24 to 50 wt. % GOS, based on carbohydrates; and/or
a weight ratio of GOS:lactose from 6:1 to 45:1.

45. The composition of claim 44, wherein the composition comprises:
from 1 to 5.5 wt. % lactose; and/or
from 30 to 48 wt. % GOS.

46. The composition of claim 44, wherein the composition comprises:
from 2.5 to 5 wt. % lactose; and/or
from 38 to 46 wt. % GOS.

47. The composition of claim 44, wherein the weight ratio of GOS:lactose is from 8:1 to 30:1

48. The composition of claim 44, wherein:
the composition further comprises galactose at a weight ratio of galactose:lactose in a range from 1.5:1 to 35:1;
the composition further comprises glucose at a weight ratio of glucose:lactose in a range from 3:1 to 40:1;
the composition further comprises from 5 to 20 wt. % galactose, based on total carbohydrates;
the composition further comprises from 15 to 30 wt. % glucose, based on total carbohydrates; or
any combination thereof.

49. The composition of claim 44, wherein the composition further comprises:
from 0.01 to 2 wt. % fat;
from 0.3 to 3 wt. % protein;
from 0.5 to 5 wt. % minerals; or
any combination thereof.
